SEQUENCE LISTING (188 sequences)

SEQ ID NO: 1 - DNA, 20 bp, Artificial
  Primer sequence
aacaaaaaatttccaccaaa

SEQ ID NO: 2 - DNA, 19 bp, artificial
  primer sequence
ttggcagagatgtgtttaa

SEQ ID NO: 3 - DNA, 18 bp, artificial
  primer sequence
caccccccaactaacaca

SEQ ID NO: 4 - DNA, 24 bp, artificial
  primer sequence
ctcccatactactaatctcatcaa

SEQ ID NO: 5 - DNA, 13 bp, artificial
  primer sequence
gggcgggggttgt

SEQ ID NO: 6 - DNA, 17 bp, artificial
  primer sequence
ttcggggtatggggtta

SEQ ID NO: 7 - DNA, 24 bp, artificial
  primer sequence
gctcttagtaagattacacatgca

SEQ ID NO: 8 - DNA, 20 bp, artificial
  primer sequence
cgtggtgatttagagggtga

SEQ ID NO: 9 - DNA, 21 bp, artificial
  primer sequence
atcaaaagggacaagcatcaa

SEQ ID NO: 10 - DNA, 18 bp, artificial
  primer sequence
taagcgttttgagctgca

SEQ ID NO: 11 - DNA, 15 bp, artificial
  primer sequence
cacccccacgggaaa

SEQ ID NO: 12 - DNA, 23 bp, artificial
  primer sequence
gttaaactttcgtttattgctaa

SEQ ID NO: 13 - DNA, 21 bp, artificial
  primer sequence
aaaaactccagttgacacaaa

SEQ ID NO: 14 - DNA, 20 bp, artificial
  primer sequence
cccagtttgggtcttagcta

SEQ ID NO: 15 - DNA, 22 bp, artificial
  primer sequence
cactccaccttactaccagaca

SEQ ID NO: 16 - DNA, 23 bp, artificial
  primer sequence
cactccaccttactaccagacaa

SEQ ID NO: 17 - DNA, 23 bp, artificial
  primer sequence
atcgcctatactttatttgggta

SEQ ID NO: 18 - DNA, 27 bp, artificial
  primer sequence
atgaattaactagaaataactttgcaa

SEQ ID NO: 19 - DNA, 18 bp, artificial
  primer sequence
ctggtttcgggggtctta

SEQ ID NO: 20 - DNA, 15 bp, artificial
  primer sequence
tgacctgcccgtgaa

SEQ ID NO: 21 - DNA, 22 bp, artificial
  primer sequence
gaccctatggagctttaattta

SEQ ID NO: 22 - DNA, 20 bp, artificial
  primer sequence
ccatagggtcttctcgtctt

SEQ ID NO: 23 - DNA, 20 bp, artificial
  primer sequence
taggacctgtgggtttgtta

SEQ ID NO: 24 - DNA, 19 bp, artificial
  primer sequence
acttcaccagtcaaagcga

SEQ ID NO: 25 - DNA, 20 bp, artificial
  primer sequence
tggtcaagttattggatcaa

SEQ ID NO: 26 - DNA, 19 bp, artificial
  primer sequence
tcgatgttggatcaggaca

SEQ ID NO: 27 - DNA, 20 bp, artificial
  primer sequence
ttaatcgttgaacaaacgaa

SEQ ID NO: 28 - DNA, 18 bp, artificial
  primer sequence
ccgccacatctaccatca

SEQ ID NO: 29 - DNA, 20 bp, artificial
  primer sequence
ggaggggggttcatagtaga

SEQ ID NO: 30 - DNA, 16 bp, artificial
  primer sequence
ccctccccatacccaa

SEQ ID NO: 31 - DNA, 21 bp, artificial
  primer sequence
tctagcctagccgtttactca

SEQ ID NO: 32 - DNA, 22 bp, artificial
  primer sequence
ggctagaggtggctagaataaa

SEQ ID NO: 33 - DNA, 19 bp, artificial
  primer sequence
gggcgtagtttgagtttga

SEQ ID NO: 34 - DNA, 25 bp, artificial
  primer sequence
cattactaataagtggctcctttaa

SEQ ID NO: 35 - DNA, 23 bp, artificial
  primer sequence
agaggtgttcttgtgttgtgata

SEQ ID NO: 36 - DNA, 22 bp, artificial
  primer sequence
ctctcccctgaactctacacaa

SEQ ID NO: 37 - DNA, 18 bp, artificial
  primer sequence
gggggtatgctgttcgaa

SEQ ID NO: 38 - DNA, 19 bp, artificial
  primer sequence
cctaccactcaccctagca

SEQ ID NO: 39 - DNA, 20 bp, artificial
  primer sequence
gggaatgctggagattgtaa

SEQ ID NO: 40 - DNA, 28 bp, artificial
  primer sequence
gataaaagagttactttgatagagtaaa

SEQ ID NO: 41 - DNA, 20 bp, artificial
  primer sequence
ggatgggttcgattctcata

SEQ ID NO: 42 - DNA, 23 bp, artificial
  primer sequence
taggcctagaaataaacatgcta

SEQ ID NO: 43 - DNA, 24 bp, artificial
  primer sequence
gagggtttatttttttggttagaa

SEQ ID NO: 44 - DNA, 23 bp, artificial
  primer sequence
ccttctaatagctatcctcttca

SEQ ID NO: 45 - DNA, 23 bp, artificial
  primer sequence
ttggtagtattggttatggttca

SEQ ID NO: 46 - DNA, 22 bp, artificial
  primer sequence
ccccatctcaatcatataccaa

SEQ ID NO: 47 - DNA, 24 bp, artificial
  primer sequence
gataagattgagagagtgaggaga

SEQ ID NO: 48 - DNA, 14 bp, artificial
  primer sequence
cggctttttgccca

SEQ ID NO: 49 - DNA, 20 bp, artificial
  primer sequence
ttttgtgaattcttcgataa

SEQ ID NO: 50 - DNA, 24 bp, artificial
  primer sequence
aaataaaatgacagtttgaacata

SEQ ID NO: 51 - DNA, 22 bp, artificial
  primer sequence
aaaggggagataggtaggagta

SEQ ID NO: 52 - DNA, 22 bp, artificial
  primer sequence
ctagacatcgtactacacgaca

SEQ ID NO: 53 - DNA, 18 bp, artificial
  primer sequence
agcctcctatgatggcaa

SEQ ID NO: 54 - DNA, 21 bp, artificial
  primer sequence
cctagaccaaacctacgccaa

SEQ ID NO: 55 - DNA, 25 bp, artificial
  primer sequence
cgtaaatctaactttcttcccacaa

SEQ ID NO: 56 - DNA, 21 bp, artificial
  primer sequence
aagttagatttacgccgatga

SEQ ID NO: 57 - DNA, 16 bp, artificial
  primer sequence
cggggcattccggata

SEQ ID NO: 58 - DNA, 20 bp, artificial
  primer sequence
cgatgcatacaccacatgaa

SEQ ID NO: 59 - DNA, 22 bp, artificial
  primer sequence
ttactgctgttagagaaatgaa

SEQ ID NO: 60 - DNA, 23 bp, artificial
  primer sequence
cctaatagtagaagaaccctcca

SEQ ID NO: 61 - DNA, 15 bp, artificial
  primer sequence
gtagggtggggggca

SEQ ID NO: 62 - DNA, 20 bp, artificial
  primer sequence
ggcctccatgactttttcaa

SEQ ID NO: 63 - DNA, 25 bp, artificial
  primer sequence
acaaagttatgaaatggtttttcta

SEQ ID NO: 64 - DNA, 21 bp, artificial
  primer sequence
cgagtagtactcccgattgaa

SEQ ID NO: 65 - DNA, 25 bp, artificial
  primer sequence
ccccattcgtataataattacatca

SEQ ID NO: 66 - DNA, 26 bp, artificial
  primer sequence
agacgtcttgtgatgtaattattata

SEQ ID NO: 67 - DNA, 21 bp, artificial
  primer sequence
gggaattgcatctgtttttaa

SEQ ID NO: 68 - DNA, 20 bp, artificial
  primer sequence
gcccatcgtcctagaattaa

SEQ ID NO: 69 - DNA, 24 bp, artificial
  primer sequence
taattcccctaaaaatctttgaaa

SEQ ID NO: 70 - DNA, 22 bp, artificial
  primer sequence
gttagctttacagtgggctcta

SEQ ID NO: 71 - DNA, 21 bp, artificial
  primer sequence
cagtgaaatgccccaactaaa

SEQ ID NO: 72 - DNA, 26 bp, artificial
  primer sequence
acccaactaaaaatattaaacacaaa

SEQ ID NO: 73 - DNA, 21 bp, artificial
  primer sequence
gggtgatgaggaatagtgtaa

SEQ ID NO: 74 - DNA, 16 bp, artificial
  primer sequence
gggctttggtgaggga

SEQ ID NO: 75 - DNA, 17 bp, artificial
  primer sequence
cattcattgcccccaca

SEQ ID NO: 76 - DNA, 20 bp, artificial
  primer sequence
gggatcaatagagggggaaa

SEQ ID NO: 77 - DNA, 22 bp, artificial
  primer sequence
tatctcatcaacaaccgactaa

SEQ ID NO: 78 - DNA, 27 bp, artificial
  primer sequence
atttgttttgaggttagtttgattagt

SEQ ID NO: 79 - DNA, 24 bp, artificial
  primer sequence
aggttcgtcctttagtgttgtgta

SEQ ID NO: 80 - DNA, 23 bp, artificial
  primer sequence
cttaatcatttttattgccacaa

SEQ ID NO: 81 - DNA, 22 bp, artificial
  primer sequence
gatagttgggtggttggtgtaa

SEQ ID NO: 82 - DNA, 25 bp, artificial
  primer sequence
ccccttatccccatactagttatta

SEQ ID NO: 83 - DNA, 18 bp, artificial
  primer sequence
ccagggctattggttgaa

SEQ ID NO: 84 - DNA, 16 bp, artificial
  primer sequence
agcgccaccctagcaa

SEQ ID NO: 85 - DNA, 25 bp, artificial
  primer sequence
acacttatcatcttcacaattctaa

SEQ ID NO: 86 - DNA, 25 bp, artificial
  primer sequence
gtgaagatgataagtgtagagggaa

SEQ ID NO: 87 - DNA, 21 bp, artificial
  primer sequence
gaaaacgtaggcttggattaa

SEQ ID NO: 88 - DNA, 23 bp, artificial
  primer sequence
gtgatttcacttccactccataa

SEQ ID NO: 89 - DNA, 22 bp, artificial
  primer sequence
cgccatcattggtatatggtta

SEQ ID NO: 90 - DNA, 15 bp, artificial
  primer sequence
gcccctaccccccaa

SEQ ID NO: 91 - DNA, 16 bp, artificial
  primer sequence
cggggtgatgcctgtt

SEQ ID NO: 92 - DNA, 25 bp, artificial
  primer sequence
ccctttctccataaaattcttctta

SEQ ID NO: 93 - DNA, 21 bp, artificial
  primer sequence
ggagggcaatttctagatcaa

SEQ ID NO: 94 - DNA, 22 bp, artificial
  primer sequence
ctaccatgagccctacaaacaa

SEQ ID NO: 95 - DNA, 20 bp, artificial
  primer sequence
agggctaggatgatgattaa

SEQ ID NO: 96 - DNA, 23 bp, artificial
  primer sequence
ctggcctatgagtgactacaaaa

SEQ ID NO: 97 - DNA, 22 bp, artificial
  primer sequence
cgaatgatttcgactcattaaa

SEQ ID NO: 98 - DNA, 22 bp, artificial
  primer sequence
gaaatcattcgttttgtttaaa

SEQ ID NO: 99 - DNA, 27 bp, artificial
  primer sequence
gaagtgagatggtaaatgctagtataa

SEQ ID NO: 100 - DNA, 23 bp, artificial
  primer sequence
cacacctcatatcctccctacta

SEQ ID NO: 101 - DNA, 21 bp, artificial
  primer sequence
tgggtgttgagggttatgaga

SEQ ID NO: 102 - DNA, 21 bp, artificial
  primer sequence
ccaatattgtgcctattgcca

SEQ ID NO: 103 - DNA, 18 bp, artificial
  primer sequence
gtgcctattgccatacta

SEQ ID NO: 104 - DNA, 23 bp, artificial
  primer sequence
ggagattgagactagtagggcta

SEQ ID NO: 105 - DNA, 23 bp, artificial
  primer sequence
tcccaacaattatattactacca

SEQ ID NO: 106 - DNA, 22 bp, artificial
  primer sequence
gtggttgtgttgattcaaatta

SEQ ID NO: 107 - DNA, 22 bp, artificial
  primer sequence
cacagcctaattattagcatca

SEQ ID NO: 108 - DNA, 21 bp, artificial
  primer sequence
aggttgttgttgatttggtta

SEQ ID NO: 109 - DNA, 14 bp, artificial
  primer sequence
gggtcggaggaaaa

SEQ ID NO: 110 - DNA, 16 bp, artificial
  primer sequence
gggggtcggaggaaaa

SEQ ID NO: 111 - DNA, 17 bp, artificial
  primer sequence
ctcccttcccctactca

SEQ ID NO: 112 - DNA, 22 bp, artificial
  primer sequence
cctagggtgttgtgagtgtaaa

SEQ ID NO: 113 - DNA, 15 bp, artificial
  primer sequence
ccatcgctgggtcaa

SEQ ID NO: 114 - DNA, 17 bp, artificial
  primer sequence
ccatagccgcctagttt

SEQ ID NO: 115 - DNA, 20 bp, artificial
  primer sequence
cggcgcagtcattctcataa

SEQ ID NO: 116 - DNA, 19 bp, artificial
  primer sequence
ggcgcagtcattctcataa

SEQ ID NO: 117 - DNA, 24 bp, artificial
  primer sequence
ggcagaatagtaatgaggatgtaa

SEQ ID NO: 118 - DNA, 20 bp, artificial
  primer sequence
gccttaccccccactattaa

SEQ ID NO: 119 - DNA, 26 bp, artificial
  primer sequence
gtaagtaggagagtgatatttgatca

SEQ ID NO: 120 - DNA, 25 bp, artificial
  primer sequence
cctctacatatttaccacaacacaa

SEQ ID NO: 121 - DNA, 26 bp, artificial
  primer sequence
gtgtgaatgagggttttatgttgtta

SEQ ID NO: 122 - DNA, 21 bp, artificial
  primer sequence
ctcgtgtgaatgagggtttta

SEQ ID NO: 123 - DNA, 23 bp, artificial
  primer sequence
agaaagctcacaagaactgctaa

SEQ ID NO: 124 - DNA, 24 bp, artificial
  primer sequence
caacatggctttctcaacttttaa

SEQ ID NO: 125 - DNA, 22 bp, artificial
  primer sequence
agttgagaaagccatgttgtta

SEQ ID NO: 126 - DNA, 23 bp, artificial
  primer sequence
gcacactactataaccaccctaa

SEQ ID NO: 127 - DNA, 21 bp, artificial
  primer sequence
gggtggttatagtagtgtgca

SEQ ID NO: 128 - DNA, 17 bp, artificial
  primer sequence
tggggggaattagggaa

SEQ ID NO: 129 - DNA, 24 bp, artificial
  primer sequence
gtgatatataaactcagacccaaa

SEQ ID NO: 130 - DNA, 25 bp, artificial
  primer sequence
gcggtaactaagattagtatggtaa

SEQ ID NO: 131 - DNA, 18 bp, artificial
  primer sequence
gctgagagggcgtaggaa

SEQ ID NO: 132 - DNA, 21 bp, artificial
  primer sequence
ggttgtataggattgcttgaa

SEQ ID NO: 133 - DNA, 20 bp, artificial
  primer sequence
ctcatgagacccacaacaaa

SEQ ID NO: 134 - DNA, 19 bp, artificial
  primer sequence
aggcttggattagcgttta

SEQ ID NO: 135 - DNA, 19 bp, artificial
  primer sequence
tcagccctactccactcaa

SEQ ID NO: 136 - DNA, 19 bp, artificial
  primer sequence
ggaagcggatgagtaagaa

SEQ ID NO: 137 - DNA, 20 bp, artificial
  primer sequence
cgtagccttctccacttcaa

SEQ ID NO: 138 - DNA, 18 bp, artificial
  primer sequence
tggttgatgccgattgta

SEQ ID NO: 139 - DNA, 17 bp, artificial
  primer sequence
cccacgccttcttcaaa

SEQ ID NO: 140 - DNA, 23 bp, artificial
  primer sequence
ttcgaatatcttgttcattgtta

SEQ ID NO: 141 - DNA, 19 bp, artificial
  primer sequence
agcctagcattagcaggaa

SEQ ID NO: 142 - DNA, 19 bp, artificial
  primer sequence
cgatgatgtggtctttgga

SEQ ID NO: 143 - DNA, 20 bp, artificial
  primer sequence
cgcctgagccctatctatta

SEQ ID NO: 144 - DNA, 20 bp, artificial
  primer sequence
cgcctatagcactcgaataa

SEQ ID NO: 145 - DNA, 21 bp, artificial
  primer sequence
gacctgttagggtgagaagaa

SEQ ID NO: 146 - DNA, 22 bp, artificial
  primer sequence
ggggttattttcgttaatgtta

SEQ ID NO: 147 - DNA, 22 bp, artificial
  primer sequence
caccctactaaaccccattaaa

SEQ ID NO: 148 - DNA, 22 bp, artificial
  primer sequence
ggggaaatgttgttagtaatga

SEQ ID NO: 149 - DNA, 18 bp, artificial
  primer sequence
cccccttccaaacaacaa

SEQ ID NO: 150 - DNA, 18 bp, artificial
  primer sequence
cgagggctgtgagtttta

SEQ ID NO: 151 - DNA, 15 bp, artificial
  primer sequence
cagcgagggctgtga

SEQ ID NO: 152 - DNA, 19 bp, artificial
  primer sequence
ccccactatgcacatttta

SEQ ID NO: 153 - DNA, 21 bp, artificial
  primer sequence
ctccaacatactcggattcta

SEQ ID NO: 154 - DNA, 20 bp, artificial
  primer sequence
ggctcgtaagaaggcctaga

SEQ ID NO: 155 - DNA, 19 bp, artificial
  primer sequence
cctgcccctactcctccta

SEQ ID NO: 156 - DNA, 19 bp, artificial
  primer sequence
tggagatttggtgctgtga

SEQ ID NO: 157 - DNA, 19 bp, artificial
  primer sequence
catcacctcaacccaaaaa

SEQ ID NO: 158 - DNA, 24 bp, artificial
  primer sequence
ggaagaagaaagagaggaagtaaa

SEQ ID NO: 159 - DNA, 22 bp, artificial
  primer sequence
ctcatcctaaccctactcctaa

SEQ ID NO: 160 - DNA, 25 bp, artificial
  primer sequence
gtagtagttactggttgaacattgt

SEQ ID NO: 161 - DNA, 17 bp, artificial
  primer sequence
tgaccccaatacgcaaa

SEQ ID NO: 162 - DNA, 17 bp, artificial
  primer sequence
catgcggagatgttgga

SEQ ID NO: 163 - DNA, 18 bp, artificial
  primer sequence
cctgcctgatcctccaaa

SEQ ID NO: 164 - DNA, 18 bp, artificial
  primer sequence
gtgggcgattgatgaaaa

SEQ ID NO: 165 - DNA, 20 bp, artificial
  primer sequence
tggcgcctcaatattcttta

SEQ ID NO: 166 - DNA, 23 bp, artificial
  primer sequence
ctgagtagagaaatgatccgtaa

SEQ ID NO: 167 - DNA, 21 bp, artificial
  primer sequence
cacacgattctttacctttca

SEQ ID NO: 168 - DNA, 20 bp, artificial
  primer sequence
tgttgctagggctgcaataa

SEQ ID NO: 169 - DNA, 21 bp, artificial
  primer sequence
ccttccacccttactacacaa

SEQ ID NO: 170 - DNA, 25 bp, artificial
  primer sequence
tctctccttaatgacattaacacta

SEQ ID NO: 171 - DNA, 24 bp, artificial
  primer sequence
gaggtctggtgagaatagtgttaa

SEQ ID NO: 172 - DNA, 19 bp, artificial
  primer sequence
ggggttggctagggtataa

SEQ ID NO: 173 - DNA, 16 bp, artificial
  primer sequence
tccgatccgtccctaa

SEQ ID NO: 174 - DNA, 21 bp, artificial
  primer sequence
ccatcctcatcctagcaataa

SEQ ID NO: 175 - DNA, 20 bp, artificial
  primer sequence
tgctaggatgaggatggata

SEQ ID NO: 176 - DNA, 16 bp, artificial
  primer sequence
ggcttagtgggcgaaa

SEQ ID NO: 177 - DNA, 17 bp, artificial
  primer sequence
tgactcctagccgcaga

SEQ ID NO: 178 - DNA, 19 bp, artificial
  primer sequence
atcggaggacaaccagtaa

SEQ ID NO: 179 - DNA, 21 bp, artificial
  primer sequence
gttgtcctccgattcaggtta

SEQ ID NO: 180 - DNA, 22 bp, artificial
  primer sequence
gctacttgtccaatgatggtaa

SEQ ID NO: 181 - DNA, 21 bp, artificial
  primer sequence
gggcctgtccttgtagtataa

SEQ ID NO: 182 - DNA, 23 bp, artificial
  primer sequence
ggagttaaagactttttctctga

SEQ ID NO: 183 - DNA, 27 bp, artificial
  primer sequence
ccacccttaacagtacatagtacataa

SEQ ID NO: 184 - DNA, 19 bp, artificial
  primer sequence
ggatgacccccctcagata

SEQ ID NO: 185 - DNA, 17 bp, artificial
  primer sequence
ctgaggggggtcatcca

SEQ ID NO: 186 - DNA, 18 bp, artificial
  primer sequence
gcactcttgtgcgggata

SEQ ID NO: 187 - DNA, 21 bp, artificial
  primer sequence
cgacatctggttcctacttca

SEQ ID NO: 188 - DNA, 17 bp, artificial
  primer sequence
ggggaacgtgtgggcta

(19) United States
(12) Patent Application Publication
(10) Pub. No.: US 2006/0078881 A1
(43) Pub. Date: Apr. 13, 2006

(54) Method and kit for detection of mutations in mitochondrial dna

(76) Inventors: Marie Allen, Uppsala (SE); Ulf Gyllensten, Uppsala (SE); Hanna Andreasson, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 10/507,873

(22) PCT Filed: Mar. 13, 2003
(86) PCT No.: PCT/SE03/00418

§ 371 Date: Sep. 19, 2005

(30) Foreign Application Priority Data
Mar. 15, 2002 (SE) 0200817-5

(51) Int. Cl. C12Q 1/68

(52) U.S. Cl. 435/6

(57) ABSTRACT

The present invention is within the medical field. More precisely, the invention relates to a method and kit for defection of mutations/polymorphisms in human mitochondrial DNA sequences and specifically to the use of mitochondrial DNA variants (polymorphisms) with high mutation frequency to be employed in the comparison of biological samples with samples of known origin in the purpose of, for example, human identification or forensic genetics.

### FIELD OF INVENTION

The present invention is within the medical field. More precisely, the invention relates to a method and kit for detection of mutations/polymorphisms in human mitochondrial DNA sequences and specifically to the use of mitochondrial DNA variants polymorphisms) to be employed in the comparison of biological samples with samples of known origin in the purpose of, for example, human identification or forensic genetics.

### BACKGROUND OF THE INVENTION

There are several methods know today for detection of mutations or polymorphisms, these can be grouped in enzymatic and non-enzymatic based methods. Non-enzymatic methods are based on hybridisation and optionally using chemical cleavage. Several patents from Affymetrix Inc., Santa Clara, Calif., USA, disclose methods where a large number of oligonucleotides are arranged on a surface, so called DNA array or DNA chips (for example, Fodor et al. U.S. Pat. No. 5,510,270). These oligonucleotide arrays are used for hybridisation of fluorescently labelled DNA and can with large number oligonucleotides, sequence and mutations can be identified. The drawback with hybridisation is that it is temperature, salt and sequence dependent and it is well known in the art that it is hard to get uniform hybridisation of many oligonucleotides at one temperature. In a situation when fluorescently labelled DNA is used as a probe the detected signal will very often differ in intensity. The generated image is then difficult to interpret and analyse.

A method called single-strand conformation polymorphism (SSCP) analysis is a intermolecular hybridisation method, where a PCR fragment is heated and quickly chilled then loaded directly on to a gel for electrophoretic separation. The drawback of this method is that they require electrophoresis, which is tedious and laboriously.

Enzymatic methods utilises an enzyme to perform the mutation detection activity, which include all sequencing methods. One method, the Enzymatic Mutation Detection technique, EMD, is a combination of hybridization and a specific heterozygote-cleaving enzyme, cleavase, this method has been developed and commercialised by Amersham Biosciences, Uppsala, Sweden. The drawback of this method is also that they require electrophoresis, which is tedious and laboriously.

Sanger sequencing or dideoxy-sequencing is the most used method for mutation discovery. A variant of this method, mini-sequencing, is used for conformation of mutations and SNP analysis. In mini-sequencing the primer is hybridised to the template just adjacent to the SNP to be studied and terminators are used in the extension reaction. The mostly used detection method is today based on dyed terminators or dyed primers, but radioactive labelled terminators or primers can also be used. Conditions and reagents for primer extension reactions are well known in the art, and are described in detail in Molecular Cloning: A laboratory manual, Sambrook et al., eds, Cold Spring Harbor Laboratory Press 1989.

Human identification has been based on analysis of either nuclear DNA or small segments of mtDNA. The studies of mtDNA, based on such materials as teeth, skeletal fragments, degraded tissue and shed hair have been focused to a small segment of the mtDNA genome, denoted the D-loop. However, in co-pending international application PCT/SE01/01691 the entire mtDNA for the purpose of human identification has been described. In this international application about 1500 polymorphic sites in the mtDNA are listed. There is no description of mutation frequency and no teachings of how to select a set of preferred polymorphic sites.

### SUMMARY OF THE INVENTION

This present invention is based on sequencing or a sequencing-by-synthesis technique and a set of primers for detection of polymorphic sites in a human mitochondrial genome. A brief description of sequencing-by-synthesis; this method was first described by Melamede and if fully described in U.S. Pat. No. 4,863,849, in short an activated nucleotide with radioactivity or a dye is added together with a DNA elongating agent to a primer-template complex and allowed to elongate. After elongation, the activated nucleotide is removed and detection is done to determine if the activated nucleotide is incorporated or not, in next step another activated nucleotide is added and elongation is allowed again, these steps are repeated until the DNA sequence of interest is determined. Different variants of sequencing-by-synthesis have been proposed, such as the Pyrosequencing™ technology developed and sold by Pyrosequencing AB, Sweden, and variants with fluorescent dyes attached to the nucleotide triphosphate at different positions.

Under specified conditions, in dideoxy-sequencing set up, when high concentrations of dideoxynucleotides are used a short stretch of DNA will be sequenced. This approach can be used in combination with the sequencing-by-synthesis primers as described in Table 1 in the present invention.

One variant of sequencing-by-synthesis is presented in U.S. Pat. No. 5,302,509 by Cheeseman, where a primer is attached to a substrate, ssDNA is hybridised thereto as a template and an added dNTP having a blocking-detectable group at the 3′-end. If the blocked-detectable dNTP is incorporated in the growing chain it can be detected, if detected the blocking group will be removed and a new blocked-detectable dNTP is added, these steps are repeated and the sequence can be deduced. Similar approaches of sequencing-by-synthesis are shown in WO 93/21340 and WO 00/53812, but here the detectable group is attached at other positions and with different linkers to the dNTP molecule. In particular WO 00/53812 disclose an array format of a sequencing-by-synthesis, which can be used in combination with the present invention. The application WO 00/53812 is hereby incorporated as a reference.

Another variant of sequencing-by-synthesis is the Pyrosequencing method, which is developed at the Royal Institute of Technology in Stockholm (Ronaghi et al. 1998, Alderborn et al. 2000). The principle of the Pyrosequencing reaction: A single stranded DNA fragment (attached to a solid support), carrying an annealed sequencing primer acts as a template for the Pyrosequencing reaction. In the first two dispensations, substrate and enzyme mixes are added to the template. The enzyme mix consists of four different enzymes; DNA polymerase, ATP-sulphurylase, luciferase and apyrase. The nucleotides are sequentially added one by one according to a specified order dependent on the template and determined by the user, this order is called dispensation order. If the added nucleotide is matching the template, the DNA polymerase will incorporate it into the growing DNA strand. By this action, pyrophosphate, PPi, will be released. The ATP-sulphurylase converts the PPi into ATP, and the third enzyme, luciferase, transforms the ATP into a light signal. Following these reactions, the fourth enzyme, apyrase, will degrade the excess nucleotides and ATP molecules, and the template will at that point be ready for next reaction cycle, i.e. another nucleotide addition. No light signal will be produced unless a correct nucleotide is incorporated. The PSQ instruments have been developed by Pyrosequencing AB in order to automate the sequencing reaction and monitor the light release. The PSQ instrument software presents the results as peaks in a pyrogram™, where the height of the peaks corresponds to the number of nucleotides incorporated. Dedicated software has been developed for SNP analysis and for sequencing of shorter DNA stretches, 20 up to 40 bases even up to 200 bases have in some situation been shown.

Compared to other techniques used for detection of polymorphic sites, such as hybridisation techniques, mini-sequencing, SSCP, sequencing-by-synthesis methods present some strong advantages. One is its ability to confirm that the correct polymorphism is examined, by presenting the surrounding sequence and not only the polymorphism/s. Another advantage is the flexibility in primer design, i.e. the primer can be situated up to 50 nucleotides from the variable site(s), where in mini-sequencing the primer has to be adjacent to the polymorphic site. Furthermore, sequencing-by-synthesis methods are rapid and direct sequencing techniques, which is benefit compare to SSCP, EMD and dideoxy-sequencing, which all requires electrophoresis a relative slow and indirect detection method.

The present invention provides a method for detection of mutations/polymorphisms in human mtDNA based on analysis of biological samples. According to the present invention, the identification is based on the analysis of genetic variation in the mitochondrial DNA (mtDNA) and comparison of the sample under investigation with that of known origin or with a database. Such analyses are useful in forensic casework, missing person identification, maternity investigations and in immigration investigations as well as in medical research.

Thus, in a first aspect the present invention relates to a method detection of mutations/polymorphisms in human mtDNA by determining the biological origin of a human tissue sample comprising the following steps:
a) determining the sequence surrounded and including the polymorphic sites having a frequency of mutation of at least 3-4% in the population according to Table 1 in the nucleic acid sequence of the mitochondrial genome in said sample from a human subject; and
a) relating the information from step a) to mitochondrial nucleic acid sequence information of known origin.

Alternative: determining polymorphisms in a certain position showing a frequency of at least 4% of the less common variant in the population according to Table 1 or showing a high frequency within the Caucasian subgroup.

The body sample referred to above can be derived from body fluid or tissue.

Preferably, also polymorphisms showing a high degree of variation within the Caucasian population are determined in step a).

In a preferred embodiment a fragment is selected such that at least one of the studied polymorphic sites has a frequency of at least 5%, preferably at least 10% and most preferably at least 15%.

The known information in step b) may be derived from human subjects of known identity (reference subjects). Alternatively, the known information in step b) is derived from a database of nucleic acid sequence information from humans of diverse origin.

In the method of the invention one or more of the mitochondrial fragments in Table 1 are selected for determination. The selected fragments are selected such that they should have at least one polymorphic site showing a frequency of at least 3-15% or harbouring polymorphisms of special interest with the Caucasian population. These fragments are 1, 4, 12, 14, 15, 16, 19, 20, 24, 25, 26, and fragment 27, according to Table 1. The fragment sizes indicated in Table are only a suggestion and can be changed according to the sequencing strategy chosen or variation frequency data obtained from a larger population set. Any forward or reverse primers (denoted F and R in Table 2) within each fragment can be combined to be the amplification primers for each fragment.

The polymorphic sites can alternatively be detected by a method or assays such as DNA hybridisation assays (ASO, SSO hybridisation, DNA microchip, padlock), enzymatic ligation assays (OLA, padlock) enzymatic cleavage assays (EMD, Taqman), enzymatic extension assays (mini-sequencing) or other assays for typing of genetic polymorphisms.

Preferably, the mitochondrial nucleic acid sequence is determined by sequencing-by-synthesis or alternatively with sequencing or preferably by a pyrosequencing technique.

The primers listed in Table 1 are preferably used. These primers have been optimized for use in the methods of the invention; it will be notable that some modification of some or all of these primers in Table 1 may be possible without adversely affecting their performance in the methods of the invention. Such modifications may be, one or more of the nucleotides, may be substituted for other (non-complementary) nucleotides. Furthermore, each primer may be expanded or deleted 1, 2, 3, 4 or even up to 5 nucleotides at the 3′ end or the 5′ end of a primer. Thus the primer can be up to 10 bases longer at the most. This can be done due to the nature of a sequencing-by-synthesis method.

In a second aspect, the present invention provides a kit for detecting the mutations/polymorphism in the human mtDNA, comprising means for analysis of the polymorphic sites having a frequency of mutation of at least 3-4% according to Table 1, preferably at least 5%, more preferably at least 10%, most preferably at least 15%.

The kit may comprise one or more of the primers in Table 1.

In a preferred kit that will perform the method of the invention the selected fragments are 1, 4, 12, 14, 15, 16, 19, 20, 24, 25, 26, and fragment 27, according to Table 1. The fragment sizes indicated in Table are only a suggestion and can be changed according to the sequencing strategy chosen. Any forward or reverse primers (denoted F and R in Table 2) within each fragment can be combined to be the amplification or sequencing primers for each fragment.

The means for analysis may be sequencing-by-synthesis reagents, sequencing reagents or pyrosequencing reagents.

I one embodiment two or more of the sequencing primers in Table 1 are attached to a solid support, such as a microtiterplate well or array. Such an array or microtiterplate with sequencing primers attached can be regarded as a component in a kit.

### DETAILED DESCRIPTION OF THE INVENTION

A Preferred Performance of the Present Invention

One hundred and thirty three polymorphic sequence sites where selected from the PCT application PCT/SE01/01691 on the basis that the frequency of the mutations should be higher than 4% in the material of 124 completely sequenced human mitochondrial genomes, some additional mutations has also been included with lower frequency, since they are informative in different populations, more specifically in a Caucasian population. These 133 mutations are located on 27 PCR fragments. The fragments are relatively short which enables analysis of degraded sample material.

Method:

One or all of the 27 PCR fragments are amplified, with two primers, where one of the primers contains means for attachment, exemplified with the streptavidin—biotin binding par. After amplification the fragment is attached to a support, which can be a solid or porous bead, a surface, such as plastic, silica or similar surface. Two, three or several DNA fragments can be attached to one surface and the can also be arrayed.

After the bind to a support one strand is removed, by temperature or high pH, at least one primer from Table 1 is annealed.

An alternative way to perform the invention is, first binding at least two sequencing primers selected from Table 1 to a solid support, secondly hybridising at least one of the amplified fragments from Table 1.

The primer in the template/primer complex is extended in a sequencing or a sequencing-by-synthesis reaction. The sequence will be generated and thereby the polymorphism will be identified.

Kit:

A kit containing amplification primers and primers as described in Table 1, for a sequencing or a sequencing-by-synthesis reaction.

A kit containing amplification and sequence primers as described in Table 1, selected is such a way that the frequency of less common variant is higher then 10%, 5%, 4% or 2%, and sequencing-by-synthesis primers as described in Table 1 for the corresponding mutations. Optionally, reagents for sequencing or sequencing-by-synthesis can be included in the kit.

TABLE 1
Polymorphic positions and frequencies

| | | No of | | | |
|---|---|---|---|---|---|
| | | p.m./124 | Polymorphism | Fragm. | Fragm. |
| Nt | Change | samples | frequency | No | Size′ |
| 316 | G −> A | 6 | 5% | 1* | |
| 456 | C −> T | 4 | 3% | 1* | |
| 462 | C −> T | 1 | 1% | 1* | |
| 489 | T −> C | 30 | 24% | 1* | |
| 514 | CA ins/del | 42 | 34% | 1* | 265 |
| 709 | G −> A | 11 | 9% | 2 | |
| 769 | G −> A | 15 | 12% | 2 | |
| 825 | T −> A | 12 | 10% | 2 | |
| 1018 | G −> A | 15 | 12% | 2 | |
| 1048 | C −> T | 7 | 6% | 2 | 399 |
| 1719 | G −> A | 7 | 6% | 3 | |
| 1888 | G −> A | 4 | 3% | 3 | 223 |
| 2706 | A −> G | 114 | 92% | 4* | |
| 2758 | G −> A | 12 | 10% | 4* | |
| 2885 | T −> C | 12 | 10% | 4* | |
| 3010 | G −> A | 13 | 10% | 4* | |
| 3027 | T −> C | 3 | 2% | 4* | 373 |
| 3516 | C −> A | 4 | 3% | 5 | |
| 3552 | T −> A | 5 | 4% | 5 | |
| 3594 | C −> T | 15 | 12% | 5 | |
| 3666 | G −> A | 7 | 6% | 5 | |
| 3796 | A −> T | 5 | 4% | 5 | 331 |
| 4104 | A −> G | 15 | 12% | 6 | |
| 4117 | T −> C | 9 | 7% | 6 | |
| 4216 | T −> C | 5 | 4% | 6 | |
| 4312 | C −> T | 5 | 4% | 6 | 302 |
| 4586 | T −> C | 5 | 4% | 7 | |
| 4715 | A −> G | 5 | 4% | 7 | |
| 4917 | A −> G | 4 | 3% | 7 | 391 |
| 5263 | C −> T | 4 | 3% | 8 | |
| 5442 | T −> C | 5 | 4% | 8 | |
| 5460 | G −> A | 15 | 12% | 8 | |
| 5465 | T −> C | 11 | 9% | 8 | 252 |
| 7028 | C −> T | 113 | 91% | 9 | |
| 7055 | A −> G | 7 | 6% | 9 | |
| 7146 | A −> G | 11 | 9% | 9 | |
| 7196 | C −> A | 5 | 4% | 9 | |
| 7256 | C −> T | 15 | 12% | 9 | |
| 7274 | C −> T | 2 | 2% | 9 | 310 |
| 7389 | T −> C | 7 | 6% | 10 | |
| 7521 | G −> A | 16 | 13% | 10 | 201 |
| 8027 | G −> A | 7 | 6% | 11 | |
| 8087 | T −> C | 4 | 3% | 11 | |
| 8251 | G −> A | 6 | 5% | 11 | |
| 8277 | Ins/Del | 19 | 15% | 11 | 304 |
| 8404 | T −> C | 8 | 6% | 12* | |
| 8414 | C −> T | 4 | 3% | 12* | |
| 8468 | C −> T | 12 | 10% | 12* | |
| 8584 | G −> A | 3 | 2% | 12* | |
| 8655 | C −> T | 12 | 10% | 12* | |
| 8697 | G −> A | 4 | 3% | 12* | |
| 8701 | A −> G | 51 | 41% | 12* | 370 |
| 8790 | G −> A | 9 | 7% | 13 | |
| 8964 | C −> T | 7 | 6% | 13 | |
| 9042 | C −> T | 5 | 4% | 13 | |
| 9072 | A −> G | 6 | 5% | 13 | |
| 9103 | T −> C | 4 | 3% | 13 | |
| 9123 | G −> A | 11 | 9% | 13 | 421 |
| 9347 | A −> G | 5 | 4% | 14* | |
| 9540 | T −> C | 51 | 41% | 14* | |
| 9545 | A −> G | 5 | 4% | 14* | 271 |
| 10238 | T −> C | 13 | 10% | 15* | |
| 10310 | G −> A | 4 | 3% | 15* | |
| 10321 | T −> C | 6 | 5% | 15* | |
| 10398 | A −> G | 55 | 44% | 15* | |
| 10400 | C −> T | 29 | 23% | 15* | |
| 10463 | T −> C | 5 | 4% | 15* | |
| 10586 | G −> A | 6 | 5% | 15* | |
| 10589 | G −> A | 5 | 4% | 15* | 420 |
| 10664 | C −> T | 5 | 4% | 16* | |
| 10688 | G −> A | 13 | 10% | 16* | |
| 10810 | T −> C | 13 | 10% | 16* | |
| 10819 | A −> G | 4 | 3% | 16* | |
| 10873 | T −> C | 50 | 40% | 16* | |
| 10915 | T −> C | 8 | 6% | 16* | 305 |
| 11251 | A −> G | 5 | 4% | 17 | |
| 11467 | A −> G | 5 | 4% | 17 | 258 |
| 11719 | G −> A | 111 | 90% | 18 | |
| 11899 | T −> C | 6 | 5% | 18 | |
| 11914 | G −> A | 16 | 13% | 18 | |
| 12007 | G −> A | 10 | 8% | 18 | 368 |
| 12239 | C −> T | 10 | 8% | 19* | |
| 12308 | A −> G | 4 | 3% | 19* | |
| 12372 | G −> A | 5 | 4% | 19* | 184 |
| 12705 | C −> T | 63 | 51% | 20* | |
| 12810 | A −> G | 6 | 5% | 20* | |
| 12940 | G −> A | 9 | 7% | 20* | 316 |
| 13105 | A −> G | 14 | 11% | 21 | |
| 13263 | A −> G | 5 | 4% | 21 | |
| 13276 | A −> G | 5 | 4% | 21 | |
| 13368 | G −> A | 5 | 4% | 21 | 343 |
| 13485 | A −> G | 6 | 5% | 22 | |
| 13500 | T −> C | 10 | 8% | 22 | |
| 13506 | C −> T | 12 | 10% | 22 | |
| 13590 | G −> A | 6 | 5% | 22 | |
| 13650 | C −> T | 15 | 12% | 22 | |
| 13708 | G −> A | 5 | 4% | 22 | |
| 13789 | T −> C | 7 | 6% | 22 | 349 |
| 13928 | G −> C | 8 | 6% | 23 | |
| 14000 | T −> A | 6 | 5% | 23 | |
| 14022 | A −> G | 10 | 8% | 23 | |
| 14025 | T −> C | 7 | 6% | 23 | |
| 14088 | T −> C | 7 | 6% | 23 | |
| 14148 | A −> G | 5 | 4% | 23 | |
| 14178 | T −> C | 7 | 6% | 23 | |
| 14182 | T −> C | 5 | 4% | 23 | 338 |
| 14766 | C −> T | 13 | 10% | 24* | |
| 14783 | T −> C | 29 | 23% | 24* | |
| 14798 | T −> C | 1 | 1% | 24* | |
| 14905 | G −> A | 6 | 5% | 24* | |
| 14911 | C −> T | 6 | 5% | 24* | |
| 15043 | G −> A | 31 | 25% | 24* | 330 |
| 15301 | G −> A | 39 | 31% | 25* | |
| 15431 | C −> A | 5 | 4% | 25* | |
| 15452 | C −> A | 5 | 4% | 25* | |
| 15487 | A −> T | 5 | 4% | 25* | 259 |
| 15607 | A −> G | 21 | 17% | 26* | |
| 15663 | T −> C | 4 | 3% | 26* | |
| 15670 | T −> C | 4 | 3% | 26* | |
| 15746 | A −> G | 10 | 8% | 26* | |
| 15784 | T −> C | 4 | 3% | 26* | |
| 15924 | A −> G | 7 | 6% | 26* | |
| 15928 | G −> A | 4 | 3% | 26* | 391 |
| 16325 | T −> C | 4 | 3% | 27* | |
| 16327 | C −> T | 6 | 5% | 27* | |
| 16343 | A −> G | 6 | 5% | 27* | |
| 16356 | T −> C | 4 | 3% | 27* | |
| 16357 | T −> C | 9 | 7% | 27* | |
| 16360 | C −> T | 8 | 6% | 27* | |
| 16362 | T −> C | 19 | 15% | 27* | |
| 16390 | G −> A | 8 | 6% | 27* | |
| 16399 | A −> G | 5 | 4% | 27* | |
| 16519 | T −> C | 69 | 56% | 27* | 259 |

′Fragment size including PCR primers.
*Denotes fragments with at least one polymorphism showing a frequency of at least 15% or harboring polymorphisms of special interest within the Caucasian population.
Numbering of mtDNA positions is according to Anderson et al. 1981.

TABLE 2

PCR and sequencing primers.
Numbers are according to Anderson et al. Primers are named by 5′ nucleotide.

| | | | | |
|---|---|---|---|---|
| Frag- | | Tm | | |
| ment | Primer | (° C.) | Sequence (5′-3′) | Nucleotides detected |

| | | | | | | | | | | | | |
|---|---|---|---|---|---|---|---|---|---|---|---|---|
| 1 | 283 F | 51,3 | AACAAAAAATTTCCACCAAA | 316 | | | | | | | | |
| 1 | 351 R | 48,6 | TTGGCAGAGATGTGTTTAA | 316 | | | | | | | | |
| 1 | 431 F | 53,6 | CACCCCCCAACTAACACA | 456 | 462 | 489 | 514 | | | | | |
| 1 | 465 F | 51,1 | CTCCCATACTACTAATCTCATC | 489 | 514 | | | | | | | |
| | | | AA | | | | | | | | | |
| 1 | 502 R | 52,0 | GGGCGGGGGTTGT | 489 | 462 | 456 | | | | | | |
| 1 | 547 R | 53,1 | TTCGGGGTATGGGGTTA | 514 | 489 | 462 | 456 | | | | | |
| 2 | 676 F | 52,3 | GCTCTTAGTAAGATTACACATGCA | 709 | 769 | | | | | | | |
| 2 | 740 R | 54,7 | CGTGGTGATTTAGAGGGTGA | 709 | | | | | | | | |
| 2 | 741 F | 55,9 | ATCAAAAGGGACAAGCATCAA | 769 | 825 | | | | | | | |
| 2 | 790 R | 52,8 | TAAGCGTTTTGAGCTGCA | 769 | 709 | | | | | | | |
| 2 | 800 F | 55,3 | CACCCCCACGGGAAA | 825 | | | | | | | | |
| 2 | 853 R | 49,8 | GTTAAACTTTCGTTTATTGCTAA | 825 | 769 | | | | | | | |
| 2 | 994 F | 51,2 | AAAAACTCCAGTTGACACAAA | 1018 | 1048 | | | | | | | |
| 2 | 1074 R | 53,3 | CCCAGTTTGGGTCTTAGCTA | 1048 | 1018 | | | | | | | |
| 3 | 1691 F-a | 53,2 | CACTCCACCTTACTACCAGACA | 1719 | | | | | | | | |
| 3 | 1691 F-b | 54,8 | CACTCCACCTTACTACCAGACAA | 1719 | | | | | | | | |
| 3 | 1752 R | 53,1 | ATCGCCTATACTTTATTTGGGTA | 1719 | | | | | | | | |
| 3 | 1856 F | 53,5 | ATGAATTAACTAGAAATAACTTTG | 1888 | | | | | | | | |
| | | | CAA | | | | | | | | | |
| 3 | 1913 R | 54,6 | CTGGTTTCGGGGGTCTTA | 1888 | | | | | | | | |
| 4 | 2680 F | 51,3 | TGACCTGCCCGTGAA | 2706 | 2758 | | | | | | | |
| 4 | 2724 F | 51,3 | GACCCTATGGAGCTTTAATTTA | 2758 | | | | | | | | |
| 4 | 2733 R | 52,2 | CCATAGGGTCTTCTCGTCTT | 2706 | | | | | | | | |
| 4 | 2782 R | 51,8 | TAGGACCTGTGGGTTTGTTA | 2758 | 2706 | | | | | | | |
| 4 | 2861 F | 52,9 | ACTTCACCAGTCAAAGCGA | 2885 | | | | | | | | |
| 4 | 2913 R | 50,1 | TGGTCAAGTTATTGGATCAA | 2885 | | | | | | | | |
| 4 | 2987 F | 54,2 | TCGATGTTGGATCAGGACA | 3010 | 3027 | | | | | | | |
| 4 | 3052 R | 50,7 | TTAATCGTTGAACAAACGAA | 3027 | 3010 | | | | | | | |
| 5 | 3493 F | 54,8 | CCGCCACATCTACCATCA | 3516 | 3552 | 3594 | | | | | | |
| 5 | 3574 R | 54,2 | GGAGGGGGGTTCATAGTAGA | 3552 | 3516 | | | | | | | |
| 5 | 3569 F | 53,2 | CCCTCCCCATACCCAA | 3594 | 3666 | | | | | | | |
| 5 | 3631 F | 53,5 | TCTAGCCTAGCCGTTTACTCA | 3666 | | | | | | | | |
| 5 | 3637 R | 53,6 | GGCTAGAGGTGGCTAGAATAAA | 3594 | 3552 | 3516 | | | | | | |
| 5 | 3691 R | 52,5 | GGGCGTAGTTTGAGTTTGA | 3666 | 3594 | | | | | | | |
| 5 | 3764 F | 52,5 | CATTACTAATAAGTGGCTCCTTT | 3796 | | | | | | | | |
| | | | AA | | | | | | | | | |
| 5 | 3823 R | 52,8 | AGAGGTGTTCTTGTGTTGTGATA | 3796 | | | | | | | | |
| 6 | 4054 F | 54,8 | CTCTCCCCTGAACTCTACACAA | 4101 | 4117 | | | | | | | |
| 6 | 4141 R | 55,5 | GGGGGTATGCTGTTCGAA | 4117 | 4101 | | | | | | | |
| 6 | 4185 F | 52,3 | CCTACCACTCACCCTAGCA | 4216 | | | | | | | | |
| 6 | 4251 R | 53,9 | GGGAATGCTGGAGATTGTAA | 4216 | | | | | | | | |
| 6 | 4275 F | 50,2 | GATAAAAGAGTTACTTTGATAGAG | 4312 | | | | | | | | |
| | | | TAAA | | | | | | | | | |
| 6 | 4355 R | 53,6 | GGATGGGTTCGATTCTCATA | 4312 | | | | | | | | |
| 7 | 4561 F | 52,1 | TAGGCGTAGAAATAAACATGCTA | 4586 | | | | | | | | |
| 7 | 4620 R | 54,1 | GAGGGTTTATTTTTTTGGTTAGAA | 4586 | | | | | | | | |
| 7 | 4676 F | 50,8 | CCTTCTAATAGCTATCCTCTTCA | 4715 | | | | | | | | |
| 7 | 4741 R | 52,9 | TTGGTAGTATTGGTTATGGTTCA | 4715 | | | | | | | | |
| 7 | 4880 F | 55,1 | CCCCATCTCAATCATATACCAA | 4917 | | | | | | | | |
| 7 | 4951 R | 52,5 | GATAAGATTGAGAGAGTGAGGAGA | 4917 | | | | | | | | |
| 8 | 5243 F | 51,3 | CGGCTTTTTGCCCA | 5263 | | | | | | | | |
| 8 | 5285 R | 47,4 | TTTTGTGAATTGTTCGATAA | 5263 | | | | | | | | |
| 8 | 5404 F | 49,6 | AAATAAAATGACAGTTTGAACATA | 5442 | 5460 | 6565 | | | | | | |
| 8 | 5494 R | 51,9 | AAAGGGGAGATAGGTAGGAGTA | 5465 | 5460 | 5442 | | | | | | |
| 9 | 6990 F | 49,4 | CTAGACATCGTACTACACGACA | 7028 | 7055 | | | | | | | |
| 9 | 7080 R | 52,7 | AGCCTCCTATGATGGCAA | 7055 | 7028 | | | | | | | |
| 9 | 7115 F | 57,9 | CCTAGACCAAACCTACGCCAA | 7146 | 7196 | | | | | | | |
| 9 | 7163 F | 56,9 | CGTAAATCTAACTTTCTTCCCAC | 7196 | 7256 | 7274 | | | | | | |
| | | | AA | | | | | | | | | |
| 9 | 7176 R | 53,1 | AAGTTAGATTTACGCCGATGA | 7146 | 7155 | | | | | | | |
| 9 | 7216 R | 57,3 | CGGGGCATTCCGGATA | 7196 | 7146 | | | | | | | |
| 9 | 7235 F | 54,9 | CGATGCATACACCACATGAA | 7256 | 7274 | | | | | | | |
| 9 | 7299 R | 48,8 | TTACTGCTGTTAGAGAAATGAA | 7274 | 7256 | 7196 | | | | | | |
| 10 | 7349 F | 52,6 | CCTAATAGTAGAAGAACCCTCCA | 7389 | | | | | | | | |
| 10 | 7409 R | 54,2 | GTAGGGTGGGGGGCA | 7389 | | | | | | | | |
| 10 | 7497 F | 56,3 | GGCCTCCATGACTTTTTCAA | 7521 | | | | | | | | |
| 10 | 7549 R | 52,5 | ACAAAGTTATGAAATGGTTTTTC | 7521 | | | | | | | | |
| | | | TA | | | | | | | | | |
| 11 | 8006 F | 53,4 | CGAGTAGTACTCCCGATTGAA | 8027 | 8087 | | | | | | | |
| 11 | 8029 F | 55,2 | CCCCATTCGTATAATAATTACAT | 8087 | | | | | | | | |
| | | | CA | | | | | | | | | |
| 11 | 8063 R | 50,5 | AGACGTCTTGTGATGTAATTATTA | 8027 | | | | | | | | |
| | | | TA | | | | | | | | | |
| 11 | 8113 R | 53,3 | GGGAATGGCATCTGTTTTTAA | 8087 | 8027 | | | | | | | |
| 11 | 8206 F | 53,2 | GCCCATCGTCCTAGAATTAA | 8251 | 8277 | | | | | | | |
| 11 | 8223 F | 54,5 | TAATTCCCCTAAAAATCTTTGAAA | 8251 | 8277 | | | | | | | |
| 11 | 8309 R | 52,2 | GTTAGCTTTACAGTGGGCTCTA | 8277 | 8251 | | | | | | | |
| 12 | 8359 F | 56,4 | CAGTGAAATGCCCCAACTAAA | 8404 | 8414 | 8468 | | | | | | |
| 12 | 8435 F | 53,8 | ACCCAACTAAAAATATTAAACACA | 8468 | | | | | | | | |
| | | | AA | | | | | | | | | |
| 12 | 8438 R | 50,4 | GGGTGATGAGGAATAGTGTAA | 8414 | 8404 | | | | | | | |
| 12 | 8488 R | 53,6 | GGGCTTTGGTGAGGGA | 8468 | 8414 | 8404 | | | | | | |
| 12 | 8549 F | 55,5 | CATTCATTGCCCCCACA | 8584 | 8655 | | | | | | | |
| 12 | 8621 R | 56,5 | GGGATCAATAGAGGGGGAAA | 8584 | | | | | | | | |
| 12 | 8632 F | 51,4 | TATCTCATCAACAACCGACTAA | 8655 | 8697 | 9701 | | | | | | |
| 12 | 8696 R | 54,4 | ATTTGTTTTGAGGTTAGTTTGATT | 8655 | 8584 | | | | | | | |
| | | | AGT | | | | | | | | | |
| 12 | 8728 R | 55,8 | AGGTTCGTCCTTTAGTGTTGTGTA | 8701 | 8697 | 8655 | | | | | | |
| 13 | 8748 F | 52,9 | CTTAATCATTTTTATTGCCACAA | 8790 | | | | | | | | |
| 13 | 8822 R | 55,2 | GATAGTTGGGTGGTTGGTGTAA | 8790 | 8701 | | | | | | | |
| 13 | 8932 F | 55,1 | CCCCTTATCCCCATACTAGTTATT | 8964 | 9042 | | | | | | | |
| | | | ATTA | | | | | | | | | |
| 13 | 8995 R | 53,1 | CCAGGGCTATTGGTTGAA | 8964 | | | | | | | | |
| 13 | 9052 F | 54,8 | AGCGCCACCCTAGCAA | 9072 | 9103 | 9123 | | | | | | |
| 13 | 9091 F | 50,9 | ACACTTATCATCTTCACAATTCT | 9123 | | | | | | | | |
| | | | AA | | | | | | | | | |
| 13 | 9107 R | 54,7 | GTGAAGATGATAAGTGTAGAGGG | 9072 | 9042 | | | | | | | |
| | | | AA | | | | | | | | | |
| 13 | 9168 R | 52,8 | GAAAACGTAGGCTTGGATTAA | 9123 | 9103 | 9072 | | | | | | |
| 14 | 9305 F | 54,6 | GTGATTTCACTTCCACTCCATAA | 8347 | | | | | | | | |
| 14 | 9382 R | 56,2 | CGCCATCATTGGTATATGGTTA | 8347 | | | | | | | | |
| 14 | 9525 F | 56,0 | GCCCCTACCCCCCAA | 9540 | 9545 | | | | | | | |
| 14 | 9575 R | 54,5 | CGGGGTGATGCCTGTT | 9545 | 9540 | | | | | | | |
| 15 | 10205 F | 55,4 | CCCTTTCTCCATAAAATTCTTCT | 10238 | 10310 | 10321 | | | | | | |
| | | | TA | | | | | | | | | |
| 15 | 10272 R | 54,7 | GGAGGGCAATTTCTAGATCAA | 10238 | | | | | | | | |
| 15 | 10281 F | 55,6 | CTACCATGAGCCCTACAAACAA | 10310 | 10321 | 10398 | 10400 | | | | | |
| 15 | 10357 R | 50,3 | AGGGCTAGGATGATGATTAA | 10321 | 10310 | 10238 | | | | | | |
| 15 | 10362 F | 55,0 | CTGGCCTATGAGTGACTACAAAA | 10398 | 10400 | 10463 | | | | | | |
| 15 | 10426 F | 53,5 | CGAATGATTTCGACTCATTAAA | 10463 | | | | | | | | |
| 15 | 10436 R | 50,6 | GAAATCATTCGTTTTGTTTAAA | 10400 | 10398 | | | | | | | |
| 15 | 10518 R | 53,6 | GAAGTGAGATGGTAAATGCTAGTA | 10463 | 10400 | 10398 | | | | | | |
| | | | TAA | | | | | | | | | |
| 15 | 10540 F | 53,8 | CACACCTCATATCCTCCCTACTA | 10586 | 10589 | | | | | | | |
| 15 | 10624 R | 56,6 | TGGGTGTTGAGGGTTATGAGA | 10589 | 10586 | | | | | | | |
| 16 | 10636 F | 57,2 | CCAATATTGTGCCTATTGCCA | 10664 | 10688 | | | | | | | |
| 16 | 10644 F | 45,3 | GTGCCTATTGCCATACTA | 10664 | 10688 | | | | | | | |
| 16 | 10715 R | 52,7 | GGAGATGAGACTAGTAGGGCTA | 10688 | 10664 | | | | | | | |
| 16 | 10776 F | 50,0 | TCCCAACAATTATATTACTACCA | 10810 | 10819 | 10873 | | | | | | |
| 16 | 10845 R | 51,6 | GTGGTTGTGTTGATTCAAATTA | 10819 | 10810 | | | | | | | |
| 16 | 10847 F | 51,5 | CACAGCCTAATTATTAGCATCA | 10873 | 10915 | | | | | | | |
| 16 | 10905 R | 51,1 | AGGTTGTTGTTGATTTGGTTA | 10873 | 10819 | 10810 | | | | | | |
| 16 | 10938 R | 45,1 | GGGTCGGAGGAAAA | 10915 | 10873 | | | | | | | |
| 16 | 10940 R | 55,2 | GGGGGTCGGAGGAAAA | 10915 | 10873 | | | | | | | |
| 17 | 11227 F | 49,0 | CTCCCTTCCCCTACTCA | 11251 | | | | | | | | |
| 17 | 11274 R | 53,2 | CCTAGGGTGTTGTGAGTGTAAA | 11251 | | | | | | | | |
| 17 | 11430 F | 50,7 | CCATCGCTGGGTCAA | 11467 | | | | | | | | |
| 17 | 11484 R | 49,9 | CCATAGCCGCCTAGTTT | 11467 | | | | | | | | |
| 18 | 11689 F | 58,5 | CGGCGCAGTCATTCTCATAA | 11719 | | | | | | | | |
| 18 | 11690 F | 53,4 | GGCGCAGTCATTCTCATAA | 11719 | | | | | | | | |
| 18 | 11744 R | 53,4 | GGCAGAATAGTAATGAGGATGTAA | 11719 | | | | | | | | |
| 18 | 11861 F | 54,5 | GCCTTACCCCCCACTATTAA | 11899 | 11914 | | | | | | | |
| 18 | 11946 R | 52,0 | GTAAGTAGGAGAGTGATATTTGAT | 11914 | 11899 | | | | | | | |
| | | | CA | | | | | | | | | |
| 18 | 11980 F | 54,1 | CCTCTACATATTTACCACAACAC | 12007 | | | | | | | | |
| | | | AA | | | | | | | | | |
| 18 | 12053 R | 57,3 | GTGTGAATGAGGGTTTTATGTTGT | 12007 | | | | | | | | |
| | | | TA | | | | | | | | | |
| 18 | 12056 R | 54,1 | CTCGTGTGAATGAGGGTTTTA | 12007 | | | | | | | | |
| 19 | 12208 F | 54,3 | AGAAAGCTCACAAGAACTGCTAA | 12239 | 12308 | | | | | | | |
| 19 | 12250 F | 56,5 | CAACATGGCTTTCTCAACTTTTAA | 12308 | 12372 | | | | | | | |
| 19 | 12268 R | 53,1 | AGTTGAGAAAGCCATGTTGTTA | 12239 | | | | | | | | |
| 19 | 12345 F | 51,9 | GCACACTACTATAACCACCCTAA | 12372 | | | | | | | | |
| 19 | 12364 R | 51,5 | GGGTGGTTATAGTAGTGTGCA | 12308 | | | | | | | | |
| 19 | 12391 R | 54,7 | TGGGGGGAATTAGGGAA | 12372 | 12308 | | | | | | | |
| 20 | 12651 F | 51,1 | GTGATATATAAACTCAGACCCAAA | 12705 | | | | | | | | |
| 20 | 12737 R | 52,4 | GCGGTAACTAAGATTAGTATGGT | 12705 | | | | | | | | |
| | | | AA | | | | | | | | | |
| 20 | 12764 F | 55,1 | GCTGAGAGGGCGTAGGAA | 12810 | | | | | | | | |
| 20 | 12862 R | 50,9 | GGTTGTATAGGATTGCTTGAA | 12810 | | | | | | | | |
| 20 | 12918 F | 53,0 | CTCATGAGACCCACAACAAA | 12940 | | | | | | | | |
| 20 | 12966 R | 51,6 | AGGCTTGGATTAGCGTTTA | 12940 | | | | | | | | |
| 21 | 13066 F | 52,8 | TCAGCCCTACTCCACTCAA | 13105 | | | | | | | | |
| 21 | 13126 R | 51,6 | GGAAGCGGATGAGTAAGAA | 13105 | | | | | | | | |
| 21 | 13239 F | 54,3 | CGTAGCCTTCTCCACTTCAA | 13263 | 13276 | | | | | | | |
| 21 | 13298 R | 52,7 | TGGTTGATGCCGATTGTA | 13276 | 13263 | | | | | | | |
| 21 | 13328 F | 54,1 | CCCACGCCTTCTTCAAA | 13368 | | | | | | | | |
| 21 | 13408 R | 52,5 | TTCGAATATCTTGTTCATTGTTA | 13368 | | | | | | | | |
| 22 | 13465 F | 51,7 | AGCCTAGCATTAGCAGGAA | 13485 | 13500 | 13506 | | | | | | |
| 22 | 13525 R | 53,8 | CGATGATGTGGTCTTTGGA | 13506 | 13500 | 13485 | | | | | | |
| 22 | 13551 F | 53,6 | CGCCTGAGCCCTATCTATTA | 13590 | 13650 | | | | | | | |
| 22 | 13596 F | 52,3 | CGCCTATAGCACTCGAATAA | 13650 | | | | | | | | |
| 22 | 13636 R | 52,2 | GACCTGTAGGGTGAGAAGAA | 13590 | | | | | | | | |
| 22 | 13680 R | 51,9 | GGGGTTATTTTCGTTAATGTTA | 13650 | 13708 | | | | | | | |
| 22 | 13680 F | 53,9 | CACCCTACTAAACCCCATTAAA | 13708 | 13789 | | | | | | | |
| 22 | 13757 R | 52,3 | GGGGAAATGTTGTTAGTAATGA | 13708 | 13650 | | | | | | | |
| 22 | 13763 F | 55,4 | CCCCCTTCCAAACAACAA | 13789 | | | | | | | | |
| 22 | 13810 R | 50,8 | CGAGGGCTGTGAGTTTTA | 13789 | 13708 | | | | | | | |
| 22 | 13813 R | 51,2 | CAGCGAGGGCTGTGA | 13789 | 13708 | | | | | | | |
| 23 | 13880 F | 50,6 | CCCCACTATGCACATTTTA | 13928 | | | | | | | | |
| 23 | 13902 F | 50,8 | CTCCAACATACTCGGATTCTA | 13928 | 14000 | 14022 | | | | | | |
| 23 | 13972 R | 55,1 | GGCTCGTAAGAAGGCCTAGA | 13928 | | | | | | | | |
| 23 | 13977 F | 55,5 | CCTGCCCCTACTCCTCCTA | 14000 | 14022 | 14025 | 14088 | | | | | |
| 23 | 14053 R | 54,5 | TGGAGATTTGGTGCTGTGA | 14025 | 14022 | 14000 | | | | | | |
| 23 | 14061 F | 53,0 | CATCACCTCAACCCAAAAA | 14088 | 14148 | 14178 | | | | | | |
| 23 | 14116 R | 53,5 | GGAAGAAGAAAGAGAGGAAGTAAA | 14088 | 14025 | 14022 | 14000 | | | | | |
| 23 | 14119 F | 51,0 | CTCATCCTAACCCTACTCCTAA | 14148 | 14178 | 14182 | | | | | | |
| 23 | 14217 R | 50,4 | GTAGTAGTTACTGGTTGAACATT | 14182 | 14178 | 14148 | | | | | | |
| | | | GT | | | | | | | | | |
| 24 | 14748 F | 52,7 | TGACCCCAATACGCAAA | 14766 | 14783 | 14798 | | | | | | |
| 24 | 14835 R | 53,0 | CATGCGGAGATGTTGGA | 14798 | 14783 | 14766 | | | | | | |
| 24 | 14862 F | 55,6 | CCTGCCTGATCCTCCAAA | 14905 | 14911 | | | | | | | |
| 24 | 14950 R | 53,9 | GTGGGCGATTGATGAAAA | 14911 | 14905 | | | | | | | |
| 24 | 15001 F | 54,8 | TGGCGCCTCAATATTCTTTA | 15043 | | | | | | | | |
| 24 | 15077 R | 52,0 | CTGAGTAGAGAAATGATCCGTAA | 10543 | | | | | | | | |
| 25 | 15271 F | 51,8 | CACACGATTCTTTACCTTTCA | 15301 | | | | | | | | |
| 25 | 15328 R | 55,8 | TGTTGCTAGGGCTGCAATAA | 15301 | | | | | | | | |
| 25 | 15402 F | 53,1 | CCTTCCACCCTTACTACACAA | 15431 | 15452 | 15487 | | | | | | |
| 25 | 15454 F | 51,1 | TCTCTCCTTAATGACATTAACAC | 15487 | | | | | | | | |
| | | | TA | | | | | | | | | |
| 25 | 15493 R | 53,3 | GAGGTCTGGTGAGAATAGTGTTAA | 15452 | 15431 | | | | | | | |
| 25 | 15529 R | 52,9 | GGGGTTGGCTAGGGTATAA | 15487 | 15452 | 15431 | | | | | | |
| 26 | 15588 F | 51,3 | TCCGATCCGTCCCTAA | 15607 | 15663 | 15670 | | | | | | |
| 26 | 15636 F | 53,2 | CCATCCTCATCCTAGCAATAA | 15663 | 15670 | 15746 | | | | | | |
| 26 | 15652 R | 51,8 | TGCTAGGATGAGGATGGATA | 15607 | | | | | | | | |
| 26 | 15710 R | 51,5 | GGCTTAGTGGGCGAAA | 15670 | 15663 | 15607 | | | | | | |
| 26 | 15722 F | 52,1 | TGACTCCTAGCCGCAGA | 15746 | | | | | | | | |
| 26 | 15758 F | 51,5 | ATCGGAGGACAACCAGTAA | 15784 | | | | | | | | |
| 26 | 15770 R | 55,4 | GTTGTCCTCCGATTCAGGTTA | 15746 | 15670 | 15663 | | | | | | |
| 26 | 15807 R | 53,3 | GCTACTTGTCCAATGATGGTAA | 15784 | 15746 | | | | | | | |
| 26 | 15882 F | 52,4 | GGGCCTGTCCTTGTAGTATAA | 15924 | 15928 | | | | | | | |
| 26 | 15978 R | 50,6 | GGAGTTAAAGACTTTTTCTCTGA | 15928 | 15924 | | | | | | | |
| 27 | 16291 F | 54,1 | CCACCCTTAACAGTACATAGTACA | 16325 | 16327 | 16343 | 16356 | 16357 | 16360 | 16362 | 16390 | 16399 |
| | | | TAA | | | | | | | | | |
| 27 | 16369 F | 55,8 | GGATGACCCCCCTCAGATA | 16390 | 16399 | | | | | | | |
| 27 | 16384 R | 56,1 | CTGAGGGGGGTCATCCA | 16362 | 16360 | 16357 | 16356 | 16343 | 16327 | 16325 | | |
| 27 | 16439 R | 54,2 | GCACTCTTGTGCGGGATA | 16399 | 16390 | 16362 | 16360 | 16357 | 16356 | 16343 | 16327 | 16325 |
| 27 | 16495 F | 54,0 | CGACATCTGGTTCCTACTTCA | 16519 | | | | | | | | |
| 27 | 16549 R | 56,3 | GGGGAACGTGTGGGCTA | 16519 | | | | | | | | |

Nucleotides detected denotes specific polymorphisms detected within 50 basepairs from the primer.

Polymorphisms shown italic denotes polymorphisms that can be detected within 100 basepairs from the primer (for fragments where longer readlengths can be obtained or when a manually programmed dispension order is used.

1. A method for detection of mutations/polymorphisms in a sample of human mitochondrial DNA comprising the following steps:
(a) determining the presence or absence of polymorphic sites having a frequency of mutation of less than 3% in the general population but at least 3% in the Caucasian population according to Table 1 in the nucleic acid sequence of the mitochondrial genome in said sample from a human subject; and
(b) relating the information from step (a) to mitochondrial nucleic acid sequence information of known origin; and
(c) relating the information from step (a), where in one or more of the mitochondrial fragments 1, 4, 12, 14, 15, 16, 19, 20, 24, 25, 26, and 27 in Table 1, to be used for determination of polymorphic site(s).

2. A method according to claim 1, wherein the frequency of mutations is at least 5%.

3. A method according to claim 1, wherein the known information in step (b) is derived from a database of nucleic acid sequence information from humans of diverse origin.

4. A method according to claim 1, wherein the polymorphic sites are detected by assays such as DNA hybridization assays (ASO, SSO hybridization, DNA microchip, padlock), enzymatic ligation assays (OLA, padlock), enzymatic cleavage assays (EMD, Taqman), enzymatic extension assays (mini-sequencing) or other assays for typing of genetic polymorphisms.

5. A method according to claim 1, wherein the mitochondrial polymorphic sites(s) is/are determined by sequencing.

6. A method according to claim 5, wherein the sequencing method is sequencing-by-synthesis.

7. A method according to claim 5, wherein the sequencing method is pyrosequencing.

8. A method according to claim 1, using the primers listed in Table 2.

9. A kit for detecting the detecting mutations/polymorphism in the human mtDNA, comprising means for analysis of the polymorphic sites having a frequency of mutation of at least 3% according to Table 1.

10. A method according to claim 9, comprising means for analysis of the polymorphic sites having a frequency of mutation of at least 5% according to Table 1.

11. A kit according to claim 9, comprising one or more of the sequencing primers in Table 2.

12. A kit according to claim 9, comprising two or more amplification primers according to Table 2 for fragment 1, 4, 12, 14, 15, 16, 19, 20, 24, 25, 26, and 27 according to Table 1.

13. A kit according to claim 9, wherein the means for analysis are sequencing-by-synthesis reagents.

14. A kit according to claim 9, wherein the means for analysis are sequencing reagents.

15. A kit according to claim 9, wherein the means for analysis are pyrosequencing reagents.

16. A kit according to claim 11, wherein two or more of the sequencing primers in Table 2 are attached to a solid support, such as a microtierplate well or array.

17. A method according to claim 1, wherein the frequency of mutations is at least 10%.

18. A method according to claim 1, wherein the frequency of mutations is at least 15%.

19. A kit according to claim 9, comprising means for analysis of the polymorphic sites having a frequency of mutation of at least 10% according to Table 1.

20. A kit according to claim 9, comprising means for analysis of the polymorphic sites having a frequency of mutation of at least 15% according to Table 1.
